(19) United States
(12) Patent Application Publication
(10) Pub. No.: US 2017/0265803 A1
(43) Pub. Date: Sep. 21, 2017

(54) LOCALIZED CANCER TUMOR DETECTION USING MICROWAVES AND NANOPARTICLES

(71) Applicant: NIMD LTD., Jerusalem (IL)

(72) Inventor: Anan COPTY, Jerusalem (IL)

(21) Appl. No.: 15/617,440

(22) Filed: Jun. 8, 2017

Related U.S. Application Data
(63) Continuation of application No. 15/533,805.
(60) Provisional application No. 62/308,248, filed on Mar. 15, 2016.

(51) Int. Cl. A61B 5/00; A61K 41/00; A61B 18/08; A61B 5/055; A61B 18/18

(52) U.S. Cl. CPC A61B 5/4836; A61B 5/4887; A61B 5/055; A61B 18/18; A61B 18/082; A61K 41/0052; A61B 2018/00702

(57) ABSTRACT

The present disclosure provides devices, systems and methods for hyperthermia cancer treatment by supplying ferromagnetic nanoparticles to a target area having or suspected of having cancer cells, the ferromagnetic nanoparticles are configured to attach to the cancer cells and heat by absorbing magnetic energy, and radiating the target area with microwaves such that the target area is within a nearfield range of the radiated microwave, and the microwave radiation nearfield is magnetically biased such that the ratio of magnetic energy to electric energy is greater than 1.

This application claims the benefit of priority of U.S. application Ser. No. 15/533,805 entitled “LOCALIZED HYPERTHERMIA/THERMAL ABLATION FOR CANCER TREATMENT”, filed on Jun. 7, 2017, which claims priority to PCT/IL2017/050327, filed on Mar. 14, 2017, which claims priority to U.S. 62/308,248 filed on Mar. 15, 2016. All applications are incorporated by reference in their entirety for all purposes.

### TECHNICAL FIELD

The present disclosure generally relates to the field of cancer treatment.

### BACKGROUND

Cancerous tissues impose a threat on the lives and wellbeing of millions of people around the globe, and in the United States alone there were an estimated 1.6 million new cases of cancer in 2015 alone, and approximately 500,000 deaths from cancer (as reported by the national cancer institute). There are currently multiple approaches for treating/managing cancer conditions in patients depending on the type of cancer and the stage thereof. Some approaches include surgery for removing the cancerous tissue, radiation therapy, chemotherapy, immunotherapy, hormone therapy, stem cell transplant and others. The efficacy of the different approaches varies, and they may be associated with side effects that include appetite loss, anemia, bleeding and bruising, Edema, fatigue, hair loss, lymphedema, peripheral neuropathy conditions, pain and more.

One approach for treating/managing cancer is called hyperthermia cancer treatment, in which the cancerous tissue is exposed to elevated temperature (above normal body temperature), to damage and kill the cancer cells, or to make the cancer cells more susceptible to other treatments such as anti-cancer drugs or ionizing radiation therapy. Common hyperthermia treatment methods include microwave (MW) or radio frequency applicators that heat cancer tissues thorough radiating the area with electromagnetic waves. The drawback to these methods is that the cancerous as well as the non-cancerous tissues surrounding the cancer cells absorb the electric energy of the electromagnetic radiation and are also heated. Therefore, besides the desired effect of damaging cancer cells, undesired damage may also occur as a result of the non-targeted heating.

There is, therefore, a need in the art for systems, methods and devices for providing an effective, accurate and selective hyperthermia cancer treatment.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

In common hyperthermia cancer treatment methods, the cancerous tissue is exposed to elevated temperature (above normal body temperature), to damage and kill the cancer cells, or to make the cancer cells more susceptible to other treatments such as anti-cancer drugs or ionizing radiation therapy. Common hyperthermia treatment methods include microwave (MW) or radio frequency applicators that heat cancer tissues through radiating the area with electromagnetic waves. One of the disadvantages to these methods is that the cancerous tissues as well as the non-cancerous tissues surrounding the cancer cells absorb the electric energy of the radiation and are also heated without selectivity. Therefore, besides the desired effect of damaging cancer cells, undesired damage may also occur as a result of the non-targeted heating.

Additionally, some of the common hyperthermia cancer treatment methods do not provide accurate control over the heating of the cancer cells. As a result, under-heating or overheating may occur. In under-heating, the temperature might not be high enough for affecting a desired treatment, and in overheating, the high temperatures of the cancer cells may stress the healthy cells and result in a negative effect. The general approach for the determination of the these conditions through a metric known as the cumulative equivalent minutes at 43° C. (CEM43° C.) where this metric depends directly on time and exponentially on temperature. The CEM43° C. is essentially a number that expresses a desired dose for a specific biological end point CEM43° C. is defined as Tᵢ.

$$CEM 43 ° C . = {\sum }_{i = 1}^{n} ti \cdot {R}^{( 43 - Ti )}$$

where tᵢ is the i-th time interval, T is the average temperature during time interval tᵢ, and R is a constant equal to 0.25 for T<43° C. and 0.5 for T>43° C. The 43° C. temperature is referring to hyperthermia treatments that are performed at or near this temperature. Thermal damage to biological tissues can occur through apoptosis or necrosis depending on the exposure time.

On top of all that, the power consumption of common hyperthermia or thermal ablation treatment systems is high (typically ranging from 40 W to 1800 W, for example, 40-100 W, 100-300 W, 150-400 W, 400-700 W, 500-1000 W, 1000-1800 W) and may require special safety considerations for providing the treatment.

According to some embodiments, the term hyperthermia, may be a general term used herein to express thermal based treatments where the temperature exceeds that of body temperatures. Typically hyperthermia denotes treatment temperatures of about 40-45° C. However, for localized hyperthermia, the temperature of the tumor may exceed 45° C. (for example, between about 45-50° C., between about 50-60° C., between about 50-70° C. or above about 70° C.) where the tumor is directly destroyed through necrosis. Thermal ablation or direct destruction of human tissue typically occurs at temperatures above 48° C. According to some embodiments, the terms hyperthermia and thermal ablation may be used interchangeably.

According to some embodiments, there are provided herein devices, systems and methods for selective hyperthermia cancer treatment and/or selective localized thermal ablation of tumors utilizing an applicator configured to provide a magnetically-biased near-field electromagnetic radiation to nanoparticles located on/in/near cancer tissue to heat the nanoparticles and thereby heat the cancer tissue. Advantageously, providing a magnetically-biased nearfield radiation at microwave frequencies to the nanoparticles may heat the cancer tissue while mitigating the heating effect of non-cancer tissues in the vicinity of the cancer tissue, as the provided radiation is characterized with a high ratio of magnetic energy to electric energy is greater than 1. The selectivity of the treatment is achieved not only by shape and dimensions of the antenna designed to heat the target tissue, but also by the selective distribution of the nanoparticles. Since the nanoparticles are configured to selectively accumulate in cancer tissue/tumors compared to normal tissue, the hyperthermia and/or localized ablation facilitates damaging the cancer tissue/tumors, while preserving or causing minimal damage to healthy surrounding tissue.

According to some embodiments, the resonance frequency of the nanoparticles is measured, and the frequency of the radiation is configured based on the resonance frequency. Advantageously, providing microwave radiation based on the resonance frequency of the nanoparticles may enable a high heating efficiency, as the same magnetic energy may result in a higher absorption in the nanoparticles.

According to some embodiments, the temperature of the cancer tissue and/or nanoparticles is measured and the radiation intensity is adjusted to prevent overheating or under-heating of the cancer tissue, advantageously resulting in a more effective treatment of the cancer cells.

In general, as the temperature of the nanoparticles changes, the resonance frequency may be affected. According to some embodiments, the frequency of the radiation may be adjusted based on the changes in temperature of the nanoparticles. Advantageously, adjusting the frequency of the radiation based on the temperature of the nanoparticles may enable high heating efficiency of the nanoparticles per given radiation intensity, and mitigate the effect of heating of surrounding non-cancer tissues.

According to some embodiments, there is provided a method for hyperthermia cancer treatment, the method includes providing ferromagnetic nanoparticles to a target area, the ferromagnetic nanoparticles are configured to absorb a magnetic field of an electromagnetic wave and thereby elevate a temperature thereof, and the ferromagnetic nanoparticles are further configured to be selectively attached to/accumulate in cancer tissue, radiating the target area with electromagnetic waves characterized with a magnetically biased nearfield behavior, wherein the distance between the target area and a source of the radiated electromagnetic waves is such that the target area is within a nearfield zone of the electromagnetic waves, thereby heat the ferromagnetic nanoparticles and the cancer tissue attached thereto, measuring reflected waves from the target area, analyzing the absorption/reflection spectrum of the target area based on the measured reflected waves compared with an absorption/reflection spectrum reference/model of the ferromagnetic nanoparticles, thereby detecting a presence (and quantity) and physical characteristics of the ferromagnetic particles in the target area, determining a temperature of the cancer tissue and/or ferromagnetic particles, and adjusting one or more parameters of the radiated electromagnetic waves based on the determined temperature of the cancer cells and/or ferromagnetic particles.

According to some embodiments, the parameters of the radiated electromagnetic waves include intensity, wavelength and/or intermittency. According to some embodiments, the parameters of the radiated electromagnetic waves are determined based on a type and characteristics of the provided ferromagnetic nanoparticles, a distance between the applicator and the target area, and/or the dimensions of the target area. According to some embodiments, the radiated electromagnetic waves have a frequency in the range of 300 MHz to 3 GHz.

According to some embodiments, the radiated electromagnetic waves have a frequency in the range of 300 MHz to 900 MHz (for example, 300-500MKz, 400-700 MHZ, 600-900 MHz). According to some embodiments, the radiated electromagnetic waves have a frequency in the range of 900 MHz to 3 GHz (for example, 1000 MHz-2GKz, 2-3 GHZ).

According to some embodiments, the distance between the target area and a source of the radiated electromagnetic waves is such that the target area is within a nearfield reactive range of the electromagnetic waves. 15

According to some embodiments, a magnetically biased nearfield behavior is characterized by a ratio of magnetic energy to electric energy greater than 1. According to some embodiments, the magnetically biased nearfield behavior is characterized by a ratio of magnetic energy to electric energy of between 2-5. According to some embodiments, the magnetically biased nearfield behavior is characterized by a ratio of magnetic energy to electric energy greater than 8.

According to some embodiments, the method further includes introducing a dielectric medium between the applicator and the target area, the dielectric medium is configured to increase the ratio of magnetic energy to electric energy of the radiation reaching the target area. According to some embodiments, the method further includes introducing a direct (non-alternating) magnetic field to the target area to enhance the efficiency of elevating the temperature of the ferromagnetic nanoparticles through resonance absorption. According to some embodiments, the direct magnetic field is in the range of 100 gauss to 4000 gauss (for example, 100 to 1000 gauss, 1000 to 2000 gauss, 1500 to 3000 gauss).

According to some embodiments, there is provided an applicator device for hyperthermia cancer treatment, the device includes an antenna configured to obtain a radiation signal, and radiate electromagnetic waves characterized with a magnetically biased nearfield behavior to a target area based on the obtained radiation signal, a magnetic energy of the electromagnetic waves is configured to be absorbed by ferromagnetic nanoparticles thereby elevate the temperature thereof, and a control circuitry configured to provide a radiation signal to the antenna, thereby define properties of the radiated electromagnetic waves, obtain a feedback signal indicative of a temperature of the ferromagnetic nanoparticles, and adjust one or more properties of the radiated electromagnetic waves based on the obtained feedback signal. Wherein the ferromagnetic nanoparticles are configured to selectively attach to cancer cells/tissue.

According to some embodiments, the device further includes a conductive plane placed adjacent to the antenna and configured to reduce/mitigate radiation not directed to the target area. According to some embodiments, the antenna includes an inductive loop. According to some embodiments, the antenna includes a flat Archimedean antenna. According to some embodiments, the antenna includes a spiral antenna. According to some embodiments, the antenna includes a small-wave antenna. According to some embodiments, the antenna includes a coaxial inductive percutaneous antenna that is configured to be placed directly inside the tumor in a minimally invasive procedure. According to some embodiments, the coaxial antennas, which may also be referred to as percutaneous antennas, may be placed directly inside a tumor, for example, a tumor having a diameter ranging from about 0.5 -2 cm.

According to some embodiments, the active area of the antenna tip may have a fixed (constant) dimensions, such as diameter. According to other embodiments, the tip maybe configured to expand upon penetrating the tumor. For example, the tip may be a coil or a helix and the diameter thereof may increase when the tip reaches the tumor. The coil or a helix may assume a pre-determined expanded form or may be controlled by a caregiver. In another example, the tip may include a bundle of antenna units and split upon reaching the tumor. In one embodiment, the coaxial antenna may be placed on an endoscope to treat deep seated tumors. In an additional or alternative embodiment, the coaxial antenna may be placed in a catheter and for delivery into the tumors.

According to some embodiments, the device further includes a collimator/lens configured to focus/direct the radiation to the target area. According to some embodiments, the device further includes a temperature sensing unit configured to measure the temperature of the ferromagnetic nanoparticles and provide the feedback signal to the control circuitry. According to some embodiments, the temperature sensing unit includes a secondary antenna, configured to measure reflected electromagnetic waves from the ferromagnetic nanoparticles. According to some embodiments, the temperature sensing unit includes a plurality fiber optic probes configured to reach to a vicinity of the target area and measure the temperature of multiple locations thereat.

According to some embodiments, there is provided a system for hyperthermia cancer treatment, the system includes ferromagnetic nanoparticles configured to be provided to a target area having or suspected of having cancer tissue, the ferromagnetic nanoparticles are configured to selectively attach to cancer tissue, the ferromagnetic nanoparticles are further configured to absorb a magnetic field of an electromagnetic wave and thereby elevate a temperature thereof, and an applicator including an antenna configured to obtain a radiation signal, and radiate electromagnetic waves characterized with a magnetically biased nearfield behavior to a target area, a magnetic energy of the electromagnetic waves is configured to be absorbed by the ferromagnetic nanoparticles thereby elevate the temperature thereof, a direct current magnetic source, configured to obtain a magnetization signal and generate a direct magnetic field in the vicinity of the target area based on the magnetization signal, thereby increase a heating efficiency of the nonmagnetic particles, and a control circuitry. According to some embodiments, the control circuitry is configured to provide the radiation signal to the antenna, thereby define properties of the radiated electromagnetic waves, provide the amount of current for the magnet, thereby define properties of the direct magnetic field, obtain a feedback signal indicative of a temperature of the ferromagnetic nanoparticles, and adjust the properties of the radiated electromagnetic waves based on the obtained feedback signal.

According to some embodiments, a magnetically biased (magnetically dominated) nearfield behavior is characterized by a ratio of a magnetic energy to an electric energy greater than 1. According to some embodiments, the system further includes a dielectric medium placed between the antenna and the target area, the dielectric medium is configured to absorb an electric energy of the radiated electromagnetic waves, thereby increase the ratio of a magnetic energy to an electric energy that reaches the target area. According to some embodiments, the direct magnetic source includes an electromagnet configured to generate a magnetic field in the vicinity of the target area.

According to some embodiments, the direct magnetic source includes at least two electromagnets placed at opposing ends/sides of the target area and configured to generate a magnetic field in the vicinity of the target area. According to some embodiments, the system further includes a magnetic modulation unit configured to modulate the magnetic field generated by the direct magnetic source. According to some embodiments, the applicator further including a conductive plane placed adjacent to the antenna and configured to reduce/mitigate radiation not directed to the target area.

According to some embodiments, there is provided a method for cancer cells detection, the method includes providing ferromagnetic nanoparticles to a target area suspected to have cancer tissue, the ferromagnetic nanoparticles configured to attach to cancer tissue, radiating the target area with electromagnetic waves characterized with a magnetically biased nearfield, measuring reflected waves from the target area, analyzing the absorption/reflection spectrum of the target area based on the measured reflected waves compared with an absorption/reflection spectrum of the provided ferromagnetic nanoparticles, thereby detect a presence of ferromagnetic particles in the target area, and determining if cancer tissue/cells are found in the target area based on the detected presence of ferromagnetic particles in the target area.

According to some embodiments, the method further includes inspecting on a shape/dimensions of a cancer tissue/tumor by performing a scanned detection of a presence of ferromagnetic particles in a plurality of segments within the target area. According to some embodiments, inspecting the shape/dimensions of a cancer tissue/tumor is a two dimensional inspection, and the plurality of segments within the target area are obtained through a planar segmentation of the target area. According to some embodiments, inspecting the shape/dimensions of a cancer tissue/tumor is a three dimensional inspection, and the plurality of segments within the target area are obtained through a volumetric segmentation of the target area.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE DRAWINGS

Examples illustrative of embodiments are described below with reference to figures attached hereto. In the figures, identical structures, elements or parts that appear in more than one figure are generally labeled with a same numeral in all the figures in which they appear. Alternatively, elements or parts that appear in more than one figure may be labeled with different numerals in the different figures in which they appear. Dimensions of components and features shown in the figures are generally chosen for convenience and clarity of presentation and are not necessarily shown in scale. The figures are listed below.

FIG. 1 schematically illustrates a system for hyperthermia cancer treatment, according to some embodiments;

FIG. 2 schematically illustrates a system for hyperthermia cancer treatment with a dielectric material, according to some embodiments;

FIG. 3 schematically illustrates a system for hyperthermia cancer treatment with temperature sensors, according to some embodiments;

FIG. 4 schematically illustrates a system for hyperthermia cancer treatment with a main antenna and a secondary antenna, according to some embodiments;

FIG. 5 schematically illustrates a functional block diagram of a system for hyperthermia cancer treatment, according to some embodiments;

FIG. 6 schematically illustrates a system for hyperthermia cancer treatment with a radiation lens/collimator, according to some embodiments;

FIG. 7 schematically illustrates a method for hyperthermia cancer treatment, according to some embodiments;

FIG. 8 illustrates resonance frequencies of an antenna, according to some embodiments;

FIG. 9a schematically illustrates a setting for measuring temperature in hyperthermia treatment, according to some embodiments;

FIG. 9b illustrates temperature measurements, according to some embodiments;

FIG. 10 illustrates temperature measurement comparison with and without a direct magnetic field, according to some embodiments;

FIG. 11 schematically illustrates a system for hyperthermia cancer treatment with a plurality of antennas, according to some embodiments;

FIG. 12 shows the median tumor volume of the control as compared to the treated group before and after the treatment; and

FIG. 13 shows the temperature profile of mouse tumor during MW ablation.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

In common hyperthermia cancer treatment methods, the cancerous tissue is exposed to elevated temperature (above normal body temperature), to damage and kill the cancer cells, or to make the cancer cells more susceptible to other treatments such as anti-cancer drugs or ionizing radiation therapy. Common hyperthermia treatment methods include microwave or radio frequency applicators that heat cancer tissues through radiating the area with electromagnetic waves. The draw back to these methods is that the cancerous as well as the non-cancerous tissues surrounding the cancer cells absorb the electric energy of the radiation and are also heated. Therefore, besides the desired effect of damaging cancer cells, undesired damage may also occur as a result of the non-targeted heating.

Additionally, some of the common hyperthermia cancer treatment methods do not provide accurate control over the heating of the cancer cells. As a result, under-heating or overheating may occur. In under-heating, the temperature might not be high enough for affecting a desired treatment, and in overheating, the high temperatures of the cancer cells may stress the healthy cells and result in a negative effect.

On top of all that, the power consumption of common hyperthermia treatment systems is high (typically ranging from 40 W to 1800 W) and may require special safety considerations for providing the treatment.

According to some embodiments, there are provided herein devices, systems and methods for Hyperthermia cancer treatment, utilizing an applicator configured to provide a magnetically-biased near-field radiation to ferromagnetic nanoparticles located on/in/near cancer tissue to heat the ferromagnetic nanoparticles and thereby heat the cancer tissue. Advantageously, providing a magnetically-biased nearfield radiation to the ferromagnetic nanoparticles may heat the cancer tissue while mitigating the heating effect of non-cancer tissues in the vicinity of the cancer tissue, as the provided radiation is characterized with a high ratio of magnetic energy to electric energy is greater than 1.

According to some embodiments, the resonance frequency of the ferromagnetic nanoparticles is measured, and the frequency of the radiation is configured based on the resonance frequency. Advantageously, providing radiation based on the resonance frequency of the ferromagnetic nanoparticles at a specific external direct magnetic field may enable a high heating efficiency, as the resonant condition may result in a higher absorption in the ferromagnetic nanoparticles to the incident microwave radiation.

According to some embodiments, the temperature of the cancer tissue and/or ferromagnetic nanoparticles is measured and the radiation intensity is adjusted to prevent overheating or undereating of the cancer tissue, advantageously resulting in a more effective treatment of the cancer cells.

In general, as the temperature of the ferromagnetic nanoparticles changes, the resonance frequency may be affected. According to some embodiments, the frequency of the radiation may be adjusted based on the changes in temperature of the ferromagnetic nanoparticles. Advantageously, adjusting the frequency of the radiation based on the temperature of the ferromagnetic nanoparticles may enable high heating efficiency of the ferromagnetic nanoparticles per given radiation intensity, and mitigate the effect of heating of surrounding non-cancer tissues.

As used herein and throughout the application, the terms “ferromagnetic nanoparticles”, “magnetic nanoparticles”, “Superparamagnetic nanoparticles” and “nanoparticles” are interchangeable, and refer to a class of particles ranging between 1 and 100 nanometers in size, and each particle behaves as a whole unit with respect to its transport and properties, and they can be manipulated using magnetic field gradients. According to some embodiments, other particle sizes may apply, such as pico-particles, micro-particles and others. According to some embodiments, the size of the ferromagnetic nanoparticles is in the range of 5 nanometer to 80 nanometer. According to some embodiments, the size of the ferromagnetic nanoparticles is in the range of 10 nanometer to 60 nanometer. According to some embodiments, the size of the ferromagnetic nanoparticles is in the range of 5 nanometer to 40 nanometer. According to some embodiments, the size of the ferromagnetic nanoparticles is in the range of 100 nanometer to 1000 nanometer.

The nanoparticles are used for tissue specific targeting, namely cancer tissue targeting, such that when they are introduced to a region with cancer tissue and other tissues, the nanoparticles will target the cancer tissue and attach thereto. Additionally, the ferromagnetic nanoparticles are designed to be manipulated by magnetic field gradients by absorbing the magnetic field energy and heating as a result, therefore, placing the ferromagnetic in an alternating/alternative magnetic field which can facilitate raising the temperature thereof.

According to some embodiments, providing the ferromagnetic nanoparticles to a target area with cancer tissue may result in having the ferromagnetic particles attaching selectively to cancer tissue and not to other tissues that may be in the target area.

When the ferromagnetic nanoparticles are heated by exposure to an alternating magnetic fields by absorbing the magnetic energy, the cancer tissue that is attached to the ferromagnetic nanoparticles is heated as well, and a hyperthermia treatment may be facilitated.

Ideally, one may want to provide magnetic fields at microwave and radio frequencies to selectively heat the cancer tissue without affecting the surrounding tissues. Practically, propagating an alternating magnetic field in space necessitates propagating an alternating electric field that is perpendicular to the magnetic field, and the magnetic field and the electric field will be perpendicular to the propagation of the resulting electromagnetic wave.

Common hyperthermia treatment methods do radiate electromagnetic waves on the target area, and heat the cancer tissue through “dielectric” heating or electric field heating, but the surrounding tissue absorbs the electric field energy and is heated as well, resulting in a non-selective heating. Different tissues may have a varying heating characteristics and therefore heat at different rates due to variations in tissue density, dielectric properties and/or thermal properties (such as thermal propagation), the variation in heating characteristics is not enough to facilitate selective heating of cancer tissues without harming surrounding tissues, and the heterogeneity of the tissues in organs makes selectivity even harder to achieve.

According to some embodiments, the target area is radiated with microwaves while positioned in the nearfield zone of the radiation, and the nearfield is characterized by inductive energy which is manifested in a greater magnetic energy than electric energy, or alternatively stated, a ratio of magnetic energy to electric energy that is greater than 1. Throughout the application, the term “magnetically biased” radiation/nearfield/waves is used to describe the property of having a magnetic energy more dominant than the electric energy in the radiated waves. As used herein, and throughout the application, the terms “magnetic energy” and “electric energy” refer to the energy of the magnetic field part or the electric field part of the incident electromagnetic wave by the microwave applicator, respectively.

Advantageously, radiating the target area with a magnetically biased nearfield radiation may facilitate a selective heating of the cancer tissue while reducing the heating of surrounding tissue that are more impacted by the electric field, and therefore may provide a hyperthermia treatment for cancer tissue while mitigating any damage that may happen to surrounding tissues resulting from the treatment.

Generally, the region that is distanced from the radiation source by more than the radiated wavelength (lambda) divided by 2 Pi (lambda/2pi) is classified as far field, while the region that is distanced from the radiation source by less than the radiated wavelength divided by 2 Pi (lambda/2pi) is classified as nearfield.

According to some embodiments, the target area is located within a reactive range of radiation, which is the non-radiative range (closest to the antenna). According to some embodiments, the antenna is designed such that in the reactive nearfield region, there is a dominance of the magnetic field (H) over the electric field (E), and the ratio between the magnetic energy and the electric energy is much greater than 1, advantageously facilitating a selective heating of the ferromagnetic nanoparticles while minimizing the heating of other (noncancerous) tissues.

According to some embodiments, the ratio between the magnetic energy and the electric energy is greater than 10. According to some embodiments, the ratio between the magnetic energy and the electric energy is greater than 20. According to some embodiments, the ratio between the magnetic energy and the electric energy is greater than 40. According to some embodiments, the ratio between the magnetic energy and the electric energy is greater than 60. According to some embodiments, the ratio between the magnetic energy and the electric energy is greater than 100.

According to some embodiments, the radiation source (antenna) is configured to radiate waves at microwave frequencies, wherein the nearfield radiation is magnetically biased. According to some embodiments, the target area with the ferromagnetic nanoparticles falls within the nearfield zone of the radiation.

According to some embodiments, the ferromagnetic nanoparticles may include Fe₃O₄, Fe₂O₃, or any combination of Fe, Co, Ni or other magnetic elements. According to some embodiments, the ferromagnetic nanoparticles may include:
Dextran or aminosilane-coated magnetite. Superparamagnetic. 3-40 nm crystal diameters.
Coated magnetite. Superparamagnetic. 10 nm and 200 nm hydrodynamic diameter.
Magnetite nanoparticles coated with lipid membrane. Administered with Interleukin-2 (IL-2).
Aminosilane-coated iron oxide. Superparamagnetic. 15 nm crystal diameter.
Dextran- and PEG-coated iron oxide, conjugated to Chimeric L6 antibody. Superparamagnetic. 20 nm hydrodynamic diameter.
Iron-based magnetic nanoparticles (10 nm crystals) loaded into liposomes. Liposomes conjugated to Trastuzumab antibody.
Ferromagnetic, dextran-coated nanoparticles. Average hydrodynamic diameter of approximately 100 nm.

According to some embodiments, Magnetite (Fe₃O₄) or other superparamagnetic particles which have a high response to a magnetic field may be utilized.

According to some embodiments, the ferromagnetic nanoparticles may be introduced to the target area by injection to veins, also known as intravenous injection, or by direct injection, also known as intra-tumoral injection, in the target area. The injection method may be selected based on the characteristics of the cancer tissue/tumor, for example, the size and irregularity thereof. According to some embodiments, the properties of the ferromagnetic nanoparticles may be selected based on the target area and/or the cancer shape and location.

According to some embodiments, the ferromagnetic nanoparticles have a diameter in the range of 5 nm to 40 nm, and advantageously in these diameters a superparamagnetic property is observed, which may increase the response to magnetic fields and increase the heating efficiency as a response.

According to some embodiments, the heating of the ferromagnetic nanoparticles is done by the ferromagnetic resonance absorption effect. The ferromagnetic resonance, or FMR, results from the magnetization of the ferromagnetic nanoparticles in the existence of an external direct magnetic field. The magnetic field is configured to utilize a torque on the magnetization, which causes precession to the magnetic moments in the ferromagnetic nanoparticles. The processing frequency of the magnetization generally depends on the orientation of the ferromagnetic nanoparticles, the strength of the magnetic field and the magnetization of the ferromagnetic nanoparticles. Unlike electron spin resonance (ESR), FMR relies on the macroscopic magnetization of the magnetic moments found in the material rather than free electrons in ESR.

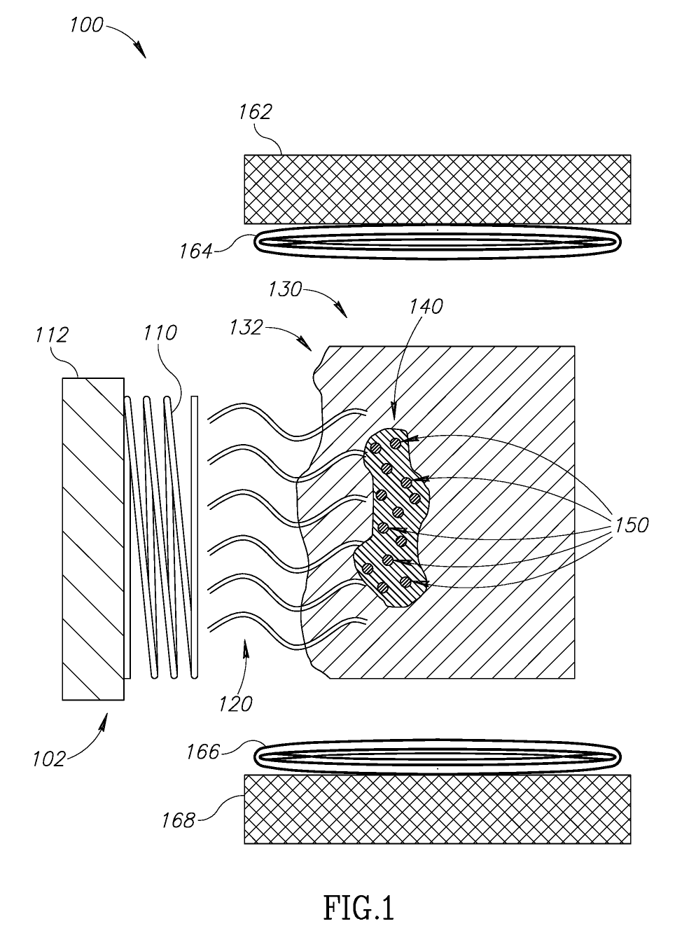

Reference is now made to FIG. 1, which schematically illustrates a system 100 for hyperthermia cancer treatment, according to some embodiments. According to some embodiments, system 100 includes an applicator 102 configured to radiate microwave radiation 120 directed to a target area 130. According to some embodiments, target area 130 has or is suspected of having a cancer tissue 140, to which a hyperthermia treatment is required. As illustrated, cancer tissue 140 is in close proximity to a skin surface 132 of target area 130, such that the cancer tissue 140 is within a nearfield range of microwave radiation 120, provided by applicator 102.

According to some embodiments, ferromagnetic nanoparticles 150 are provided to target area 130, and configured to selectively attach to cancer tissue 140. According to some embodiments, ferromagnetic nanoparticles 150 are configured to heat by absorbing magnetic energy, which is part of the microwave radiation 120 which is characterized with a dominance of magnetic energy compared to electric energy, thereby facilitating an effective heating of ferromagnetic nanoparticles 150 (and cancer tissue 140, as a result).

According to some embodiments, applicator 102 includes an antenna 110 configured to radiate microwave radiation 120, and a conductive plane 112 configured to obstruct radiation from antenna 110 that is not directed towards target area 130. According to some embodiments, the dimensions of antenna 110 are associated with the size and dimensions of cancer tissue 140. According to some embodiments, the dimensions of antenna 110 are configured to be larger than a cross section of cancer tissue 140. According to some embodiments, antenna 110 is shaped approximately 5 cm in length and 5 cm in width. According to some embodiments, antenna 110 is shaped approximately 2 cm in length and 2 cm in width.

According to some embodiments, the length and/or width of antenna 110 is in the range of 1 cm to 10 cm. According to some embodiments, the length and/or width of antenna 110 is in the range of 2 cm to 8 cm. According to some embodiments, the length and/or width of antenna 110 is in the range of 2 cm to 30 cm. According to some embodiments, the length and/or width of antenna 110 is in the range of 2 cm to 5 cm.

According to some embodiments, antenna 110 is configured to radiate microwave radiation 120 with a frequency ranging from 300 MHz to 300 GHz with corresponding wavelengths of 100 cm to 0.1 cm respectively. According to some embodiments, antenna 110 is configured to radiate microwave radiation 120 with a high frequency ranging from 0.3 GHz to 30 GHz with corresponding wavelengths of 100 cm to 1 cm respectively.

According to some embodiments, antenna 110 is configured to radiate microwave radiation 120 with a frequency ranging from 0.3 GHz to 3 GHz with corresponding wavelengths of 100 cm to 10 cm respectively.

As described earlier, the region of the nearfield corresponds with the wavelength (lambda), such that for a further nearfield region reach a longer wavelength may be utilized, and for a closer nearfield region reach a shorter wavelength may be utilized.

According to some embodiments, antenna 110 is configured to radiate microwave radiation 120 with a frequency ranging from 300 MHz to 900 MHz with corresponding wavelengths of 100 cm to 33 cm respectively, to achieve a deep penetration/reach of the nearfield region, and to target cancer cells that are approximately 7 cm to 0.6 cm under the skin surface depending on the dielectric property of the tissue. According to some embodiments, antenna 110 is configured to radiate microwave radiation 120 with a frequency ranging from 900 MHz to 3 GHz with corresponding wavelengths of 33 cm to 10 cm respectively, to achieve a shallow penetration/reach of the nearfield region, and to target cancer cells that are approximately 2.5 cm to 0.1 cm under the skin surface.

An antenna is designed to radiate at directions based on the shape and properties in which it was configured, but radiation to other directions may be unavoidable by antenna design. For example, an antenna designed to radiate to one direction (front) will have a “front lobe”/“main lobe” signifying the radiation intensity to the front direction, but may also have a non-negligible “back lobe” which signifies the radiation intensity to the opposite direction. It is beneficial to minimize and/or avoid this radiation which is not directed towards the target area.

According to some embodiments, conductive plane 112 is placed behind antenna 110 and configured to absorb and/or reflect the “back lobe” radiation that is directed away from the target area. Advantageously, obstructing the radiation to an undesired area facilitates safety and efficiency. According to some embodiments, the distance between the back plane (conductive plane 112) and the antenna is determined by the radiated wavelength and is approximately the wavelength divided by 10.

According to some embodiments, system 100 further includes a first and a second direct (DC) magnetic sources, such as first electromagnet 162 and second electromagnet 168, which are configured to generate a direct magnetic field in an area comprising cancer tissue 140 and ferromagnetic nanoparticles 150. According to some embodiments, providing a direct magnetic field to ferromagnetic nanoparticles 150 increases and/or decreases the absorption thereof to the magnetic energy of microwave radiation 120 based on the strength of the direct magnetic field, and increases and/or decreases the heating efficiency accordingly.

According to some embodiments, system 100 further includes a first magnetic field modulator such as first coil 164 and a second magnetic field modulator such as second coil 166, adjacent to first electromagnet 162 and second electromagnet 168 respectively and configured to modulate the direct magnetic field provided to the target area. According to some embodiments, in the detection mode, the modulating coils are used to enhance the signal to noise ratio of the detected signal when coupled with a lock-in amplifier.

According to some embodiments, the direct magnetic field provided to the target area is in the range of 100 Gauss to 4000 Gauss. According to some embodiments, a direct magnetic field of less than 3000 Gauss is utilized for increasing the heating efficiency, while a direct magnetic field of more than 3000 Gauss is utilized for decreasing the heating efficiency depending on the applied microwave frequency of the applicator. According to some embodiments, the magnetic field modulators are configured to control the intensity of the provided direct magnetic field for increasing and/or decreasing the heating efficiency. According to some embodiments, the direct magnetic field used for increasing the efficiency of the heating is in the range of 200 Gauss to 800 Gauss. According to some embodiments, the direct magnetic field used for increasing the efficiency of the heating is in the range of 400 Gauss to 800 Gauss. According to some embodiments, the direct magnetic field used for increasing the efficiency of the heating is approximately 500 Gauss.

According to some embodiments, system 100 is configured to operate applicator 102 and the other components thereof for heating cancer tissue 140 to approximately 42 degrees Celsius. According to some embodiments, system 100 is configured to operate applicator 102 and the other components thereof for heating cancer tissue 140 to a temperature in the range of 41 to 43 degrees Celsius. According to some embodiments, system 100 is configured to operate applicator 102 and the other components thereof for heating cancer tissue 140 to a temperature in the range of 40 to 45 degrees Celsius. According to some embodiments, system 100 is configured to operate applicator 102 and the other components thereof for heating cancer tissue 140 to ablative temperatures in the range of 45 to 55 degrees Celsius. According to some embodiments, system 100 is configured to operate applicator 102 and the other components thereof for heating cancer tissue 140 to ablative temperatures of 55 degrees Celsius. According to some embodiments, system 100 is configured to operate applicator 102 and the other components thereof for heating cancer tissue 140 to ablative temperatures in the range of 50 to 55 degrees Celsius. According to some embodiments, system 100 is configured to operate applicator 102 and the other components thereof for heating cancer tissue 140 to ablative temperatures in the range of 50 to 90 degrees Celsius.

If heat is applied to the cancer tissue not at the desired treatment temperature, the treatment may not be effective (in the case of under heating), or may stress the healthy tissue and result in undesired effects in the case of overheating. According to some embodiments, to achieve the precise/accurate heating to the desired temperature, a feedback signal indicative of the temperature of the cancer tissue is obtained, and the radiation and/or direct magnetic field are adjusted accordingly for accurately reaching the treatment temperature.

According to some embodiments, it is desired to heat the cancer tissue to the target/treatment temperature at a predetermined rate, and the heating rate may also be achieved by means of temperature sensing (via the signal indicative of cancer tissue temperature) and adjusting the radiation and/or properties of direct magnetic field accordingly. In some embodiments, it is desired to have a heating rate of above 2 degree-Celsius per minute, as having a low heating rate may result in heat propagating to neighboring tissues to which the hyperthermia or thermal ablation is not required or even harmful. For performing localized thermal ablation where the temperature of the heated tumor exceeds 50 degrees-Celsius, it is desirable to have a fast heating rate of 6-30 degrees per minute such that the tumor is ablated rapidly without allowing enough time for the high temperature to transfer to the surrounding tissue.

According to some embodiments, the heating rate may be determined by the specific absorption rate (SAR) parameter, which is indicative of the microwave energy absorption rate per volume or tissue. Therefore, utilizing nanoparticles with high SAR may facilitate a high efficiency and selectivity of heating.

According to some embodiments, the magnetic field modulators and/or the direct magnetic sources may include an electromagnet, a coil/solenoid, a static magnet the like and/or any combination thereof.

According to some embodiments, the antenna in the applicator may include an inductive loop antenna, a flat Archimedean antenna, a spiral antenna, a small-wave antenna, a coaxial inductive antenna or the like and/or any combination thereof.

The characteristics of the radiation are determined by the type and shape of the antenna, the local environment being irradiated as well as the provided radiation signal which is provided by a driver/controller.

According to some embodiments, the antenna is an Archimedean spiral antenna shaped as a two-armed spiral having a minimal spiral radius and a maximal spiral radius. According to some embodiments, the minimal spiral radius dictates the maximal frequency that the antenna can achieve (radiate), while the maximal spiral radius dictates the minimal frequency that the antenna can achieve.

The frequency limitation may be as follows:

The highest frequency is dictated by the minimal spiral radius such that:

$${f}_{h} = \frac{c}{2 π {r}_{min}}$$

The highest frequency is dictated by the minimal spiral radius such that:

$${f}_{l} = \frac{c}{2 π {r}_{max}}$$

And other radiation characteristics may be determined by the shape and density of the loops. For instance, a more homogeneous and high filed density radiation may be obtained from an antenna having more loops.

According to some embodiments, an effectiveness of heating of the ferromagnetic nanoparticles or the cancer tissue is determined by the amount of energy required to heat the cancer tissue to a desired temperature. Advantageously, when the nearfield radiation that reaches the ferromagnetic nanoparticles is predominantly magnetic, more of the antenna energy is utilized for heating the cancer tissue than dielectric heating of surrounding tissues due to the presence of the nanoparticles inside the cancer tissue. As a result, the operation power of the applicator may be reduced compared to common hyperthermia devices.

According to some embodiments, the operation power of the applicator is in the range of 0.1 Watt to 100 Watts. According to some embodiments, the operation power of the applicator is in the range of 0.5 Watt to 20 Watts. According to some embodiments, the operation power of the applicator is in the range of 1 Watt to 10 Watts. According to some embodiments, the operation power of the applicator is in the range of 2 Watts to 5 Watts. According to some embodiments, the operation power of the applicator is approximately 4 Watts.

According to some embodiments, a dielectric material/medium (or highly absorbing dielectric material/medium) may be introduced between the applicator/antenna and the target area. The dielectric medium is configured to absorb the electric energy produced by the antenna such that the radiation reaching the target area is more magnetically biased with less electric energy than otherwise. Advantageously, absorbing the electric energy (at least partially) may mitigate the risk of heating non-cancerous tissues from the electric energy due to dielectric heating.

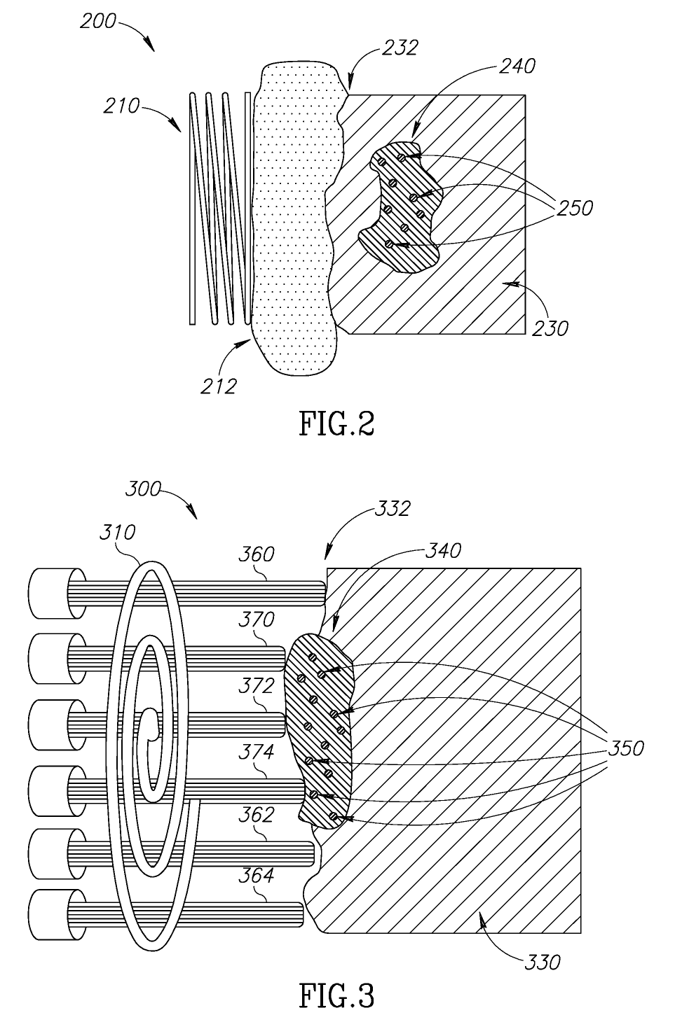

Reference is now made to FIG. 2, which schematically illustrates a system 200 for hyperthermia cancer treatment with a dielectric medium 212, according to some embodiments. According to some embodiments, a small-wave antenna 210 is configured to radiate microwave radiation directed to a target area 230 with a cancer tissue 240, and ferromagnetic nanoparticles 250 are introduced to target area 230 and configured to selectively attach to cancer cells in cancer tissue 240. Ferromagnetic nanoparticles are configured to heat by absorbing magnetic energy from the radiated microwave radiation, while other parts of target area 230 may heat from the electric energy of the radiated microwave radiation via dielectric heating. According to some embodiments, a dielectric medium 212 is introduced between small-wave antenna 210 and skin/surface 232 of target area 230, and is configured to at least partially absorb the electric energy of the radiated microwave radiation while allowing the magnetic energy to pass through without significantly absorbing it, thereby minimizing/mitigating the heating of non-cancer tissues from dielectric heating and increasing the heating selectivity (heating the cancer tissue only).

According to some embodiments, the dielectric medium or highly absorbent dielectric medium may include: Ethylene Glycol, Ethyl Alcohol (absolute), distilled water, or others. It is important that the medium used does not contain metallic elements as these elements are highly absorbing of magnetic fields due to the creation of “Eddy” currents from the inductive fields in the metal.

According to some embodiments, a temperature measuring mechanism/unit may be utilized for measuring the temperature of the cancer tissue and/or the surrounding tissues. According to some embodiments, the radiation and/or direct current is modulated based on the temperature measurements to achieve an accurate heating of the cancer tissue to the desired target/treatment temperature, without damaging the surrounding tissues.

According to some embodiments, the temperature sensing mechanism may include contact and non-contact techniques such as: infrared cameras, fiber optics, a bimetal sensor, an integrated circuitry temperature transducer, a thermocouple sensor or the like or any combination thereof.

Reference is now made to FIG. 3, which schematically illustrates a system 300 for hyperthermia cancer treatment with a temperature sensing mechanism, according to some embodiments. According to some embodiments, a spiral antenna 310 is configured to radiate microwave radiation directed to a target area 330 with a cancer tissue 340, and ferromagnetic nanoparticles 350 are introduced to target area 330 and configured to selectively attach to cancer cells in cancer tissue 340. Ferromagnetic nanoparticles are configured to heat by absorbing magnetic energy from the radiated microwave antenna. According to some embodiments, system 300 further includes a temperature sensing mechanism, such as fiber-optic sensors 360, 362, 364, 370, 372 and 374 that are configured to be slide-able/movable to reach the skin surface 332 of target area 330 and measure the temperature at various points thereat.

According to some embodiments, optic-fiber sensors 360, 362 and 364 are configured to measure the temperature indicative of the heating of non-cancer tissues, while optic-fiber sensors 370, 372 and 374 are configured to measure the temperature indicative of the heating of cancer tissue 340. According to some embodiments measuring the temperature may enable heating cancer tissue 340 to the desired target/treatment temperature while not harming surrounding tissues.

According to some embodiments, the radiating antenna or a second antenna may be utilized for measuring the temperature of the cancer tissue by measuring the resonance frequencies of the ferromagnetic nanoparticles. The resonance frequencies of the ferromagnetic nanoparticles is temperature dependent, and drifts (changes) with changes of temperature. According to some embodiments, measuring the resonance temperature of the ferromagnetic nanoparticles is indicative of the temperature thereof. Therefore, measuring the resonance frequency accurately with each temperature point, can provide noninvasive temperature information about the heated tumor. Moreover, since FMR resonance frequency is only related to the nanoparticles and not the tissue, only the temperature of the tumor will be detected. For an accurate evaluation of the temperature, the optimization of the signal strength detected by the microwave probe is critical. The signal, usually measured in its derivative form will be curve fitted for accurate evaluation of the resonance frequency and will always be compared to the initial reference signal at body temperature (that is 37 degrees-Celsius). A temperature calibration curve is used in practice.

According to some embodiments, when the temperature of the ferromagnetic nanoparticles is known, or for this matter the resonance frequency, the radiation wavelength may be adjusted accordingly to “lock” on the resonance frequency and maintain a high heating efficiency.

According to some embodiments, the absorption/reflection spectrum of the target area based on the measured reflected waves compared with an absorption/reflection spectrum reference/model of the ferromagnetic nanoparticles, thereby detecting a presence and physical characteristics of the ferromagnetic particles in the target area. According to some embodiments, the absorption/reflection spectrum model/reference is a sample or aggregation of samples of absorption spectra for the ferromagnetic nanoparticles at one selected temperature, or at different temperature values. And as the resonance absorption of the ferromagnetic nanoparticles drifts/changes with temperature variation, the examined spectrum or behavior may be compared with the model to detect the temperature of the ferromagnetic nanoparticles.

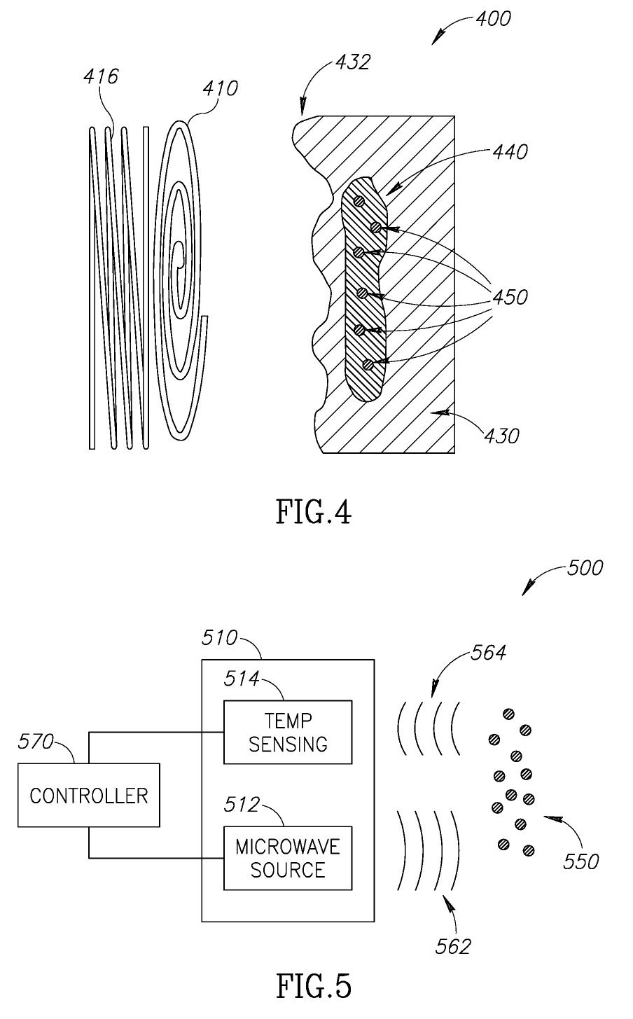

Reference is now made to FIG. 4, which schematically illustrates a system 400 for hyperthermia cancer treatment with a main antenna 410 and a secondary antenna 416, according to some embodiments. According to some embodiments, main antenna 410 is a spiral antenna and is configured to radiate microwave radiation directed to a target area 430 with a cancer tissue 440, and ferromagnetic nanoparticles 450 are introduced to target area 430 and configured to selectively attach to cancer cells in cancer tissue 440. Ferromagnetic nanoparticles are configured to heat by absorbing magnetic energy from the radiated microwave radiation, while other parts of target area 430 may heat from the electric energy of the radiated microwave radiation via dielectric heating. According to some embodiments, secondary antenna 416 is configured to measure the resonance frequencies of ferromagnetic nanoparticles 450 to measure the temperature thereof and/or for providing feedback to adjust the radiation frequency of main antenna 410 based on the detected resonance frequencies.

According to some embodiments, a single antenna may be utilized for both radiating to heat the ferromagnetic nanoparticles and for measuring/detecting the resonance frequencies thereof by intermittent operation. The intermittent operation is such that at certain times the antenna operates for heating the ferromagnetic nanoparticles, while at other times the antenna operates for detecting the resonance frequencies. According to some embodiments, the heating periods may last up to a few minutes (for example up to 6 minutes), and are separated by resonance detection periods of up to 5 seconds.

According to some embodiments, the heating periods may last up to 60 minutes at hyperthermia temperatures of 40-43 degrees-Celsius, for 6-15 minutes at ablative 10 temperatures of 55 degrees-Celsius and 2-6 minutes at 65 degrees-Celsius, and are separated by resonance detection periods of up to 5-30 seconds. Eventually, a pre-determined CEM43° C. value will govern the thermal dose.

Reference is now made to FIG. 5, which schematically illustrates a functional block diagram of a system 500 for hyperthermia cancer treatment, according to some embodiments. According to some embodiments, system 500 includes an applicator 510 with a temperature sensing mechanism 514 and a microwave source 512 such as an antenna, and further includes a controller 570 configured to operate microwave source 512, for radiating microwave radiation 562 to heat a plurality of ferromagnetic nanoparticles 550. According to some embodiments, temperature sensing mechanism 514 is configured to provide controlled 570 with a signal indicative of a temperature of ferromagnetic nanoparticles 550, and controller 550 is further configured to adjust the operation of microwave source 512 based on the provided signal.

According to some embodiments, in order to focus the radiation towards the target area, a collimator or lens may be used, the collimator may be made of Teflon or Alumina. According to some embodiments, the shape of the lens is concave, such that the emitted microwave beam from the applicator is converged to focus the beam on the target area.

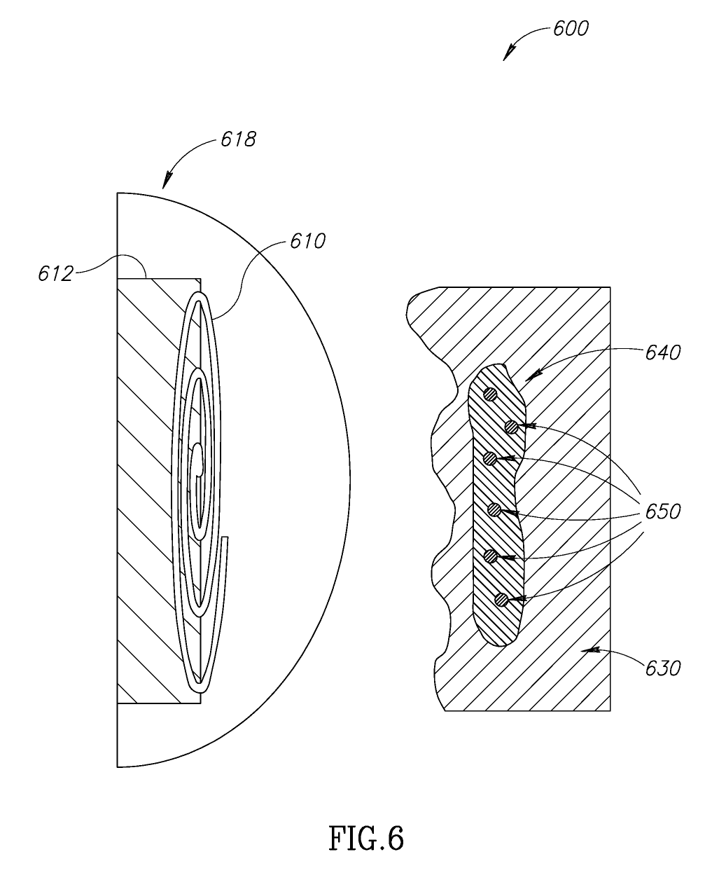

Reference is now made to FIG. 6, which schematically illustrates a system 600 for hyperthermia cancer treatment with a radiation lens/collimator 618, according to some embodiments. According to some embodiments, system 600 includes a spiral antenna 610 configured to radiate microwaves towards a target area 630 having therein a cancer tissue 640 with ferromagnetic particles 650 provided thereto and configured to selectively attach to cancer tissue 640. Ferromagnetic nanoparticles 650 are configured to heat by absorbing the magnetic energy of the radiated microwave beam. According to some embodiments, a back-plane 612 is provided for absorbing the radiation that is directed distally from target area 630, and collimator 618 is configured to focus the radiation towards target area 630, thereby facilitating better selectivity and efficiency.

According to some embodiments, the collimator may be at least partially made of a dielectric material, and configured to both focus the radiation towards the target area and to absorb at least parts of the radiated electric energy to facilitate higher selectivity and efficiency.

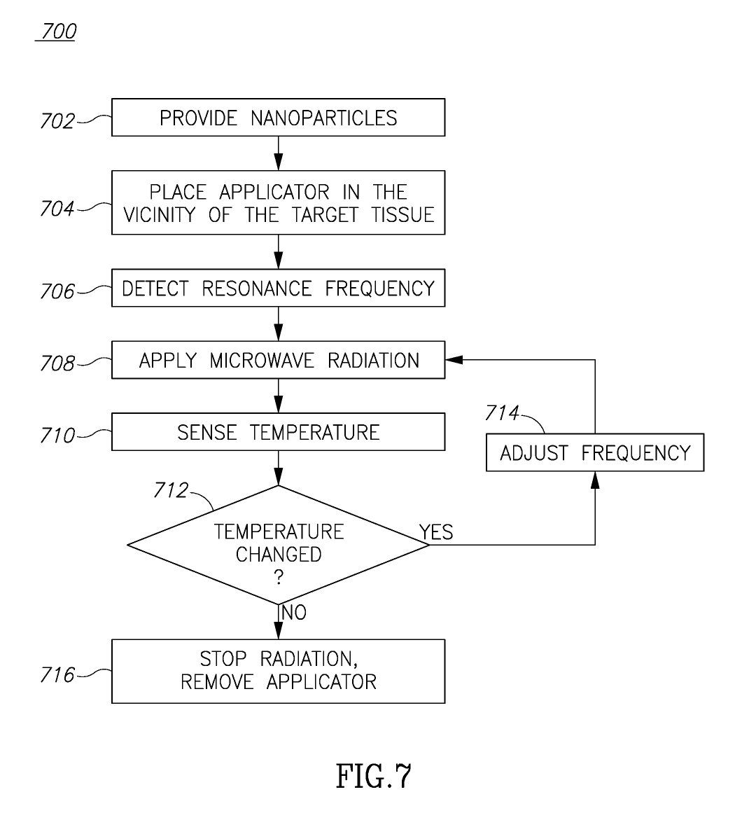

Reference is now made to FIG. 7, which schematically illustrates a method 700 for hyperthermia cancer treatment, according to some embodiments. According to some embodiments, method 700 begins with providing ferromagnetic nanoparticles to a target area with cancer tissue (step 702), then an applicator is placed in the vicinity of the target tissue (step 704), then the resonance frequency (or frequencies) are detected and/or analyzed (step 706) and microwave radiation is applied for heating the ferromagnetic nanoparticles (step 708). Afterwards, the temperature of the ferromagnetic particles and/or cancer tissue may be sensed (step 710) and based on the sensed temperature (step 712) the radiation properties may be adjusted (step 714) and when the treatment is over, the radiation is terminated/stopped and the applicator is removed (step 716).

According to some embodiments, the methods, systems and devices provided herein may be utilized for detection of cancer cells/tissues, for example by providing the ferromagnetic nanoparticles to a suspected target area, and radiating to detect a resonance frequency and/or absorption pattern associated with the ferromagnetic nanoparticles, and if ferromagnetic nanoparticles were detected, this may indicate that cancer cells/tissues are found in the target area.

According to some embodiments, an antenna with multiple resonance frequencies may provide a wider range of operation, by enabling radiation at a wider frequency range.

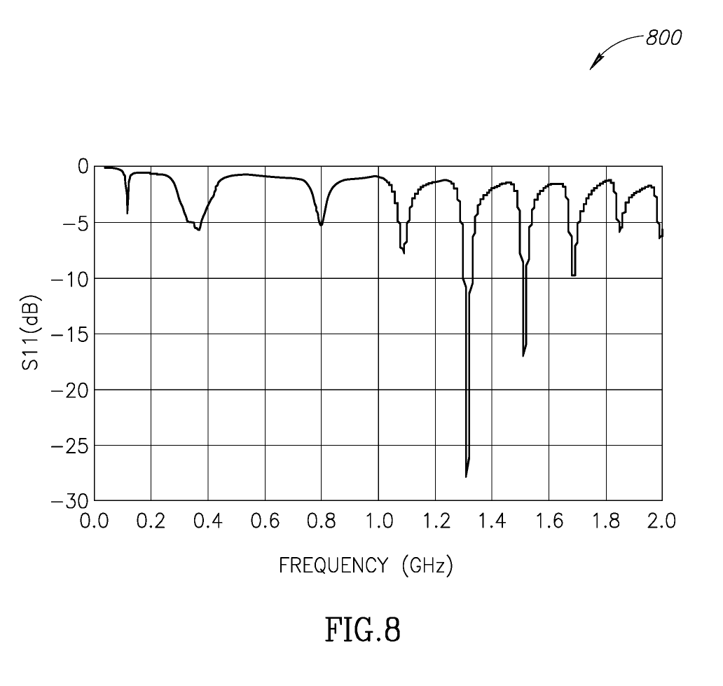

Reference is now made to FIG. 8, which illustrates resonance frequencies 800 of a small-wave antenna, according to some embodiments. Advantageously, the small-wave antenna provides multiple resonance frequencies, resulting in a wide operational range of ˜200 MHz to ˜2 GHz.

### Experiments

One of the experiments on a device as described herein was designed to measure the selectivity and efficiency of heating.

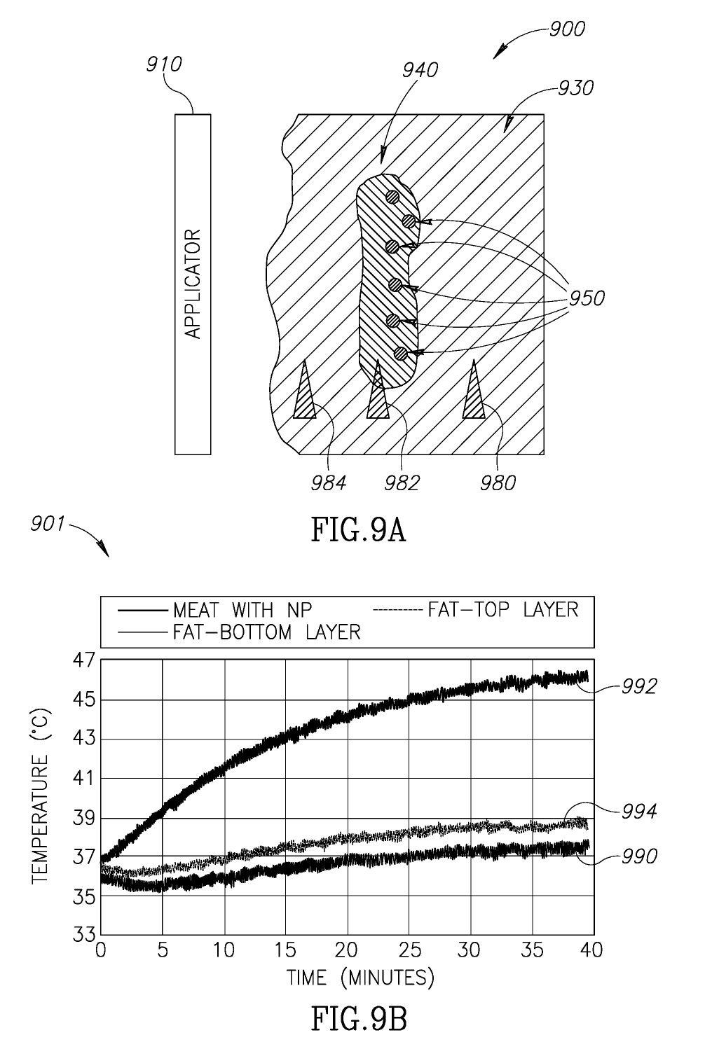

Reference is now made to FIG. 9a, which schematically illustrates a setting 900 for measuring temperature in hyperthermia treatment, with a device according to some embodiments. As experimented, an applicator 910 was configured to radiate microwave radiation towards a mammal tissue target area 930 with a cancer tissue 940 having ferromagnetic nanoparticles 950 attached thereto for absorbing the magnetic energy of the radiated microwave radiation. Additionally, a first temperature sensing probe 980 was introduced for measuring the temperature within target area 930 near cancer tissue 940 further from applicator 910, a second temperature sensing probe 982 was introduced for measuring the temperature of cancer tissue 940 and a third temperature sensing probe 984 was introduced for measuring the temperature within target area 930 near cancer tissue 940 closer to applicator 910.

Reference is now made to FIG. 9b, which illustrates a graph 901 temperature measurements, obtained in the experiment. As shown, while the temperatures of non-cancer tissues distally and proximally to the applicator, 990 and 994 respectively, have not changed significantly, the temperature of the cancer tissue 992 has gone up to the desired operation/target temperature of approximately 46 degrees Celsius.

This demonstrates an exceptional selectivity and heating efficiency, which is advantageous for a targeted cancer heating without significantly harming the surrounding tissues and/or organs, or even without harming them at all.

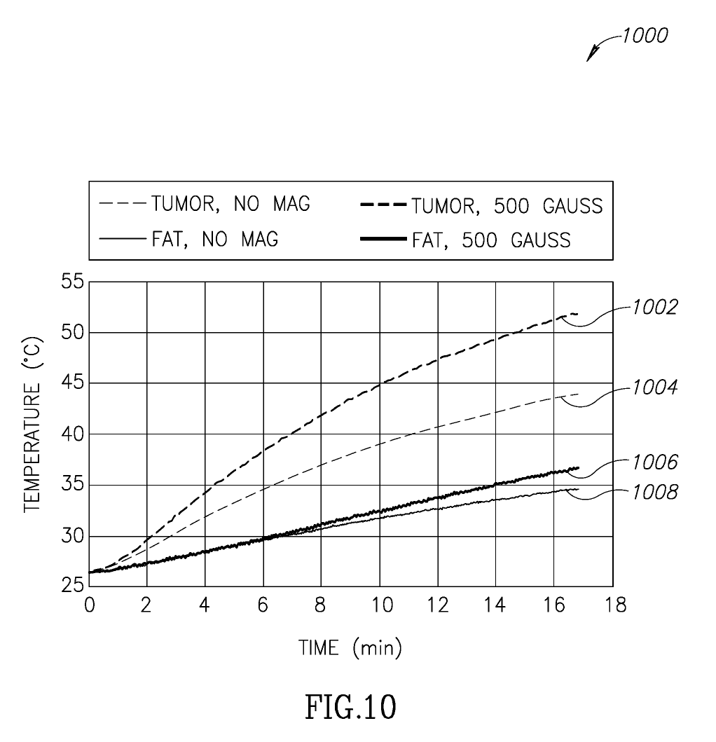

Reference is now made to FIG. 10, which illustrates a graph 1000 of temperature measurement comparison with and without a direct external magnetic field, experimentally, according to some embodiments. As shown, the temperature of a non-cancer tissue (fat) was measured with and without a direct magnetic field, 1006 and 1008, respectively, and the direct magnetic field had an insignificant effect on the heating of this tissue, while measuring the temperature of the cancer tissue with and without the direct magnetic field, 1002 and 1004 respectively, shows that the direct magnetic field has a significant effect on the heating of the cancer tissue, which may increase the efficiency of the heating and reduce the power consumption of the overall operation. It is to be noted that the direct magnetic field applied in this experiment was approximately 500 Gauss.

According to some embodiments, the antenna may include a plurality of antennas, for example an array of antennas. Advantageously, utilizing an array of antennas may enable providing hyperthermia treatment to large cancer tumors/tissues.

According to some embodiments, the intensity of the antennas may not be uniform, and different antennas may be configured to radiate at different radiation intensities and/or frequencies. Advantageously, as the cancer tissue may not be homogeneously distributed, especially, when the tumor area is large or there is a collection of tumors found in a specific area, or the ferromagnetic nanoparticles may not attach homogeneously across the cancer tissue, it may be desired to radiate differently to different segments/areas of the cancer tissue. It is generally desired to heat the whole tissue to the target temperature, and practically, some segments of the tissue may heat faster (at a higher rate) than other segments, therefore, according to some embodiments, the antennas associated-with or targeting those segments may be adjusted to adjust the temperature to achieve a balanced heating of the cancer tissue across the different segments thereof.

As the cancer tissue is generally irregular, different segments may have different depths under the skin/surface. According to some embodiments, different antennas may radiate at different frequencies based on the depth of the corresponding cancer tissue segment, advantageously providing uniform/homogeneous heating of the cancer tissue compensating on depth differences of the cancer tissue segments.

Additionally, the radiation intensities are generally not uniform across the area of the antenna; therefore the radiation effect of one antenna is not uniform. Advantageously, using a plurality of antennas may facilitate a more uniform behavior of the radiation in different locations.

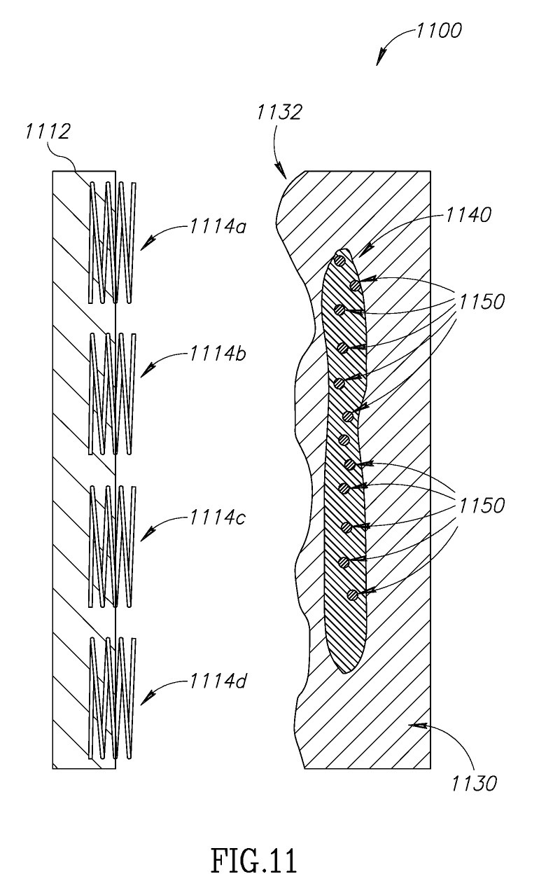

Reference is now made to FIG. 11, which schematically illustrates a system 1100 for hyperthermia cancer treatment with an array of antennas 1114a-d, according to some embodiments. According to some embodiments, antennas 1114a-d are configured to radiate microwave radiation towards a target area 1130 having a cancer tissue 1140 with ferromagnetic nanoparticles 1150 configured to attach thereto, According to some embodiments, behind antennas 1114a-d (distally from target area), there is a conductive plane 1112 configured to absorb radiation directed distally from target area 1130, and the operation of each antennas 1114a-d may be independent in intensity, frequency or both.

According to some embodiments, a plurality of antennas may be utilized for targeting a target area or a segment thereon, the antennas having different characteristics, for example different frequency ranges, and may be selectively operated based on the required radiation frequency.

According to some embodiments, a two-dimensional scanning or a three-dimensional scanning may be performed using the devices and systems provided herein. According to some embodiments, a scanner may be utilized for moving the antenna/applicator on X-Y axes, and the resonance frequencies may be measured on multiple points in the plane for detecting cancer tissue and generate a scanning image of the cancer cell. According to some embodiments, a scanner may be utilized for moving the antenna/applicator on X-Y-Z axes, and the resonance frequencies and intensities may be measured within the volume to create a three-dimensional scanning of the cancer tissue.

According to some embodiments, a post-surgery cancer detection or assessment may be performed using the devices and systems provided herein.

Generally, for post-surgery applications, it is often very difficult for the operating surgeon to determine whether the cancer has been completely removed or not. Typically, surgeons may add a margin of a few millimeters or more beyond the physical tumor to ensure all cancer cells have been removed. But, currently they do not have a practical method to detect residual tumors post-surgery. According to some embodiments, as the nanoparticles attach selectively to cancer cells and can be detected through the use of the FMR technique, they can give indicators for cancer detection including early cancer detection as well as post-surgery evaluation. By enhancing the microwave probe detection sensitivity and with the use of highly magnetic nanoparticles it is possible to detect cancer with very few cells.

According to some embodiments, types of cancer that may be targeted include amongst others: skin cancer, breast cancer, lung cancer, head & neck cancers, chest wall, axilla, glioblastoma or any solid tumor.

According to some embodiments, pre-cancerous tumors such as actinic keratosis may be targeted.

According to some embodiments, the applicator device, system and methods may apply to cancer on/near the skin surface in human subject or other mammals, Canaan dogs for example.

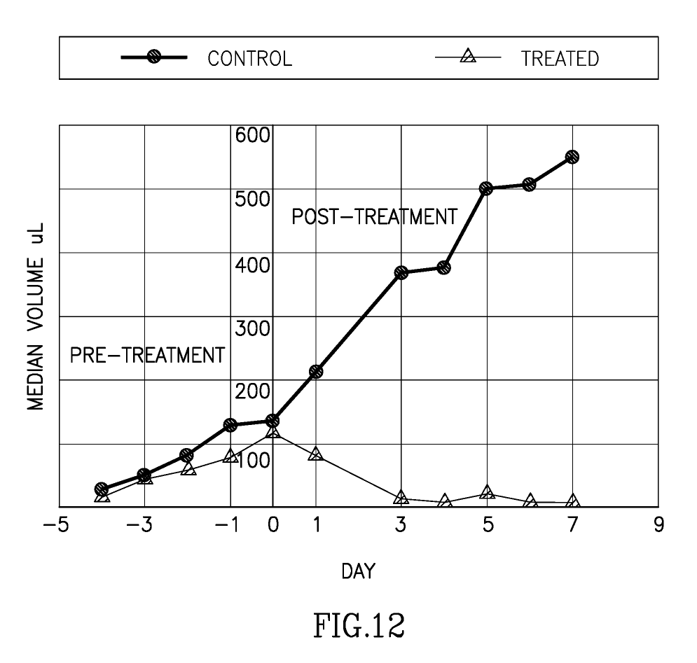

Mice experiments were also performed and demonstrated the efficiency of the herein disclosed method and system. In the experiment, 16 female mice, 6 weeks old, BALB/C, that were subcutaneously injected with 4T1 cells to induce tumor growth, were subjected to the thermal treatment disclosed herein. In short, once the tumors reached an average size of 0.5-0.6 cm diameter, 50 μL nanoparticles having a density of 2 mg Fe/ml and a an average particle diameter of approximately 30 nm were injected directly into the tumor. 12 hours after injection of the nanoparticles, the mice were either left untreated (n=8) or treated (n=8) using a slow-wave 2.5 cm×2.5 cm antenna. The applied power was 8 Watts and the thermal dose was controlled using the CEM43° C. metric. FIG. 12 shows the median tumor volume of the control mice as compared to the treated mice, before and after the treatment. As demonstrated, the tumor volume of the treated group greatly diminished already 3 days after the treatment, whereas the tumor size in the control mice continued to increase.

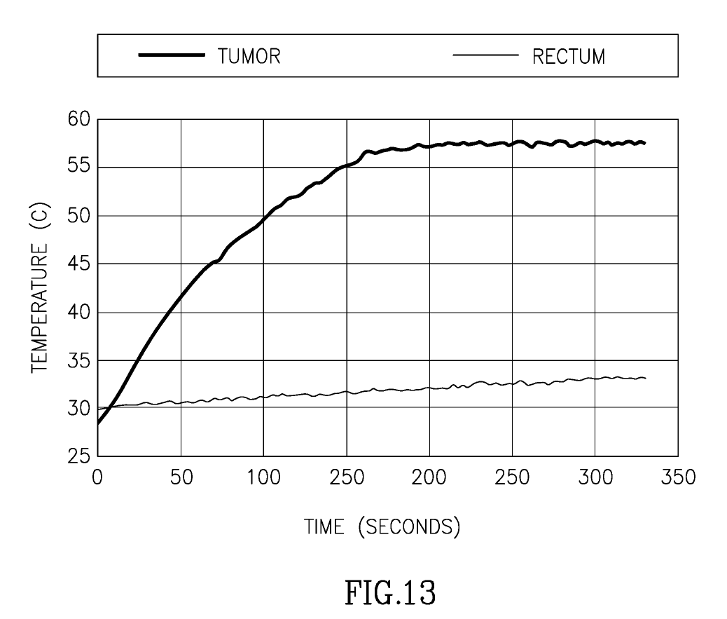

FIG. 13 shows the temperature profile of mouse tumor during MW ablation. Optical fiber thermal sensors were placed inside the tumor and at the rectum of the mice to quantify the body temperature. As seen from FIG. 13, the temperature of the tumor reached the pre-set 57° C. temperature after about 160 seconds. Nevertheless, rectum temperature increased by no more than 3-4° C. from its original temperature during the entire treatment, thus, demonstrating that the heating process is selectively damaging the tumor tissue, while the body temperature of the mouse, despite it weighing only about 20 grams, remains very close to its normal body temperature.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” or “comprising,” when used in this specification, specify the presence of stated features, integers, steps, operations, elements, or components, but do not preclude or rule out the presence or addition of one or more other features, integers, steps, operations, elements, components, or groups thereof.

While a number of exemplary aspects and embodiments have been discussed above, those of skill in the art will recognize certain modifications, additions and sub-combinations thereof. It is therefore intended that the following appended claims and claims hereafter introduced be interpreted to include all such modifications, additions and sub-combinations as are within their true spirit and scope.

What we claim is:

1. A method for cancer cells/tumor detection, the method comprising:
providing ferromagnetic nanoparticles to a target area suspected to have cancer tissue, the ferromagnetic nanoparticles configured to attach to cancer tissue;
utilizing an applicator comprising an antenna, radiating the target area with electromagnetic waves characterized with a magnetically biased nearfield behavior, the electromagnetic waves having a frequency in the microwave range, a magnetic energy of the electromagnetic waves is configured to be absorbed by the ferromagnetic nanoparticles;
utilizing a direct current magnetic source, obtaining a magnetization signal and generating a direct magnetic field in the vicinity of the target area based on the magnetization signal;
measuring reflected waves from the target area;
analyzing the absorption/reflection spectrum of the target area based on the measured reflected waves compared with an absorption/reflection spectrum of the provided ferromagnetic nanoparticles, thereby detect a presence of ferromagnetic particles in the target area; and
determining if cancer tissue/cells are found in the target area based on the detected presence of ferromagnetic particles in the target area.

2. The method of claim 1, wherein the distance between the target area and a source of the radiated electromagnetic waves is such that the target area is within a nearfield reactive range of the electromagnetic waves.

3. The method of claim 1, wherein the magnetically biased nearfield radiation behavior is characterized by a ratio of a magnetic energy to an electric energy greater than 1.

4. The method of claim 1, wherein the magnetically biased nearfield radiation behavior is characterized by a ratio of a magnetic energy to an electric energy greater than 8.

5. The method of claim 1, further comprising introducing a dielectric medium between an antenna and a skin surface of the target area, the dielectric medium is configured to absorb an electric energy of the radiated electromagnetic waves, thereby increase the ratio of a magnetic energy to an electric energy that reaches the target area.

6. The method of claim 1, the microwave radiation has a frequency ranging from 300 MHz to 900 MHz, to target cancer cells, within a nearfield region, that are approximately 7 cm to 0.6 cm under the skin surface.

7. The method of claim 1, the microwave radiation has a frequency ranging from 900 MHz to 3 GHz, to target cancer cells, within a nearfield region, that are approximately 2.5 cm to 0.1 cm under the skin surface.

8. The method of claim 1, further comprising inspecting on a shape/dimensions of a cancer tissue/tumor by performing a scanned detection of a presence of ferromagnetic particles in a plurality of segments within the target area.

9. The method of claim 8, wherein inspecting the shape/dimensions of a cancer tissue/tumor is a two dimensional inspection, and the plurality of segments within the target area are obtained through a planar segmentation of the target area.

10. The method of claim 8, wherein inspecting the shape/dimensions of a cancer tissue/tumor is a three dimensional inspection, and the plurality of segments within the target area are obtained through a volumetric segmentation of the target area.

11. The method of claim 1, wherein the antenna comprises an inductive loop.

12. The method of claim 1, wherein the antenna comprises a flat Archimedean antenna.

13. The method of claim 1, wherein the antenna comprises a spiral antenna.

14. The method of claim 1, wherein the antenna comprises a small-wave antenna.

15. The method of claim 1, wherein the direct magnetic source comprises an electromagnet configured to generate a magnetic field in the vicinity of the target area.

16. The method of claim 1, wherein the direct magnetic source comprises at least two electromagnets, the method further comprising placing the at least two electromagnets at opposing ends/sides of the target area and generating a magnetic field in the vicinity of the target area.

17. The method of claim 1, further comprising, utilizing a magnetic modulation unit comprising coils, modulating the magnetic field generated by the direct magnetic source.

18. The method of claim 1, further comprising placing a conductive plane adjacent to the antenna and thus reducing/mitigating radiation not directed to the target area.

19. A system for cancer cells/tissue detection, the system comprising:
an applicator comprising an antenna configured to obtain a radiation signal, and radiate electromagnetic waves characterized with a magnetically biased nearfield behavior to a target area, a magnetic energy of the electromagnetic waves is configured to be absorbed by ferromagnetic nanoparticles, the electromagnetic waves having a frequency in the microwave range, the ferromagnetic nanoparticles are configured to selectively attach to cancer cells/tissue and to absorb a magnetic field of an electromagnetic wave;
a direct current magnetic source, configured to obtain a magnetization signal and generate a direct magnetic field in the vicinity of the target area based on the magnetization signal; and
a control circuitry configured to:
provide the radiation signal to said antenna, thereby define properties of the radiated electromagnetic waves;
provide the amount of current for the magnet, thereby define properties of the direct magnetic field;
analyze the absorption/reflection spectrum of the target area based on the measured reflected waves compared with an absorption/reflection spectrum of the provided ferromagnetic nanoparticles, thereby detect a presence of ferromagnetic particles in the target area; and
determining if cancer tissue/cells are found in the target area based on the detected presence of ferromagnetic particles in the target area.
